(19) United States
(12) Patent Application Publication
(10) Pub. No.: US 2014/0378917 A1
(43) Pub. Date: Dec. 25, 2014

(54) TRANSDERMAL THERAPEUTIC SYSTEM WITH A LOW TENDENCY TO SPONTANEOUSLY CRYSTALLIZE

(71) Applicants: UCB Pharma GmbH, Monheim (DE); LTS LOHMANN THERAPIE SYSTEME AG, Andernach (DE)

(72) Inventors: Horst Dzekan, Melnbom (DE); Hans-Rainer Hoffmann, Neuwied (DE); Michael Horstmann, Neuwied (DE); Walter Mueller, Andernach (DE); Sandra Wiedersberg, Steigra (DE)

(21) Appl. No.: 14/368,864

(22) PCT Filed: Dec. 28, 2012
(86) PCT No.: PCT/EP2012/077046

§ 371 Date: Jun. 26, 2014

(30) Foreign Application Priority Data
Dec. 30, 2011 (DE) 10 2011 090 178.7

(51) Int. Cl. A61K 9/70; A61K 31/381; A61K 31/496; A61K 31/5375; A61K 31/439; A61K 31/485; A61K 31/325

(52) U.S. Cl. CPC A61K 9/7053; A61K 9/7069; A61K 31/485; A61K 31/381; A61K 31/325; A61K 31/5375; A61K 31/439; A61K 31/496
USPC 604/307; 427/2.31; 424/448; 514/279; 514/438; 514/490; 514/230.5; 514/299; 514/254.06

(57) ABSTRACT

The present invention provides transdermal therapeutic systems for dispensing a pharmaceutical product, in particular a pharmaceutical product with a low tendency to spontaneously crystallize. The matrix layer or at least one of the matrix layers in said systems contains a silicone hot-melt adhesive, a pharmaceutical product, and particles made of crosslinked polyvinylpyrrolidone. The invention also relates to a method for producing said transdermal therapeutic systems.

The present application claims priority from PCT Patent Application No. PCT/EP2012/077046 filed on Dec. 28, 2012, which claims priority from German Patent Application No. DE 10 2011 090 178.7 filed on Dec. 30, 2011, the disclosures of which are incorporated herein by reference in their entirety.

The present invention relates to transdermal therapeutic systems for delivering drugs, said transdermal therapeutic systems having a low tendency for spontaneous crystallisation of the drug contained therein.

It is noted that citation or identification of any document in this application is not an admission that such document is available as prior art to the present invention.

Transdermal therapeutic systems (TTS) have been known for several years and are meanwhile established on the pharmaceutical market. There are transdermal therapeutic systems which contain the drug in dissolved form in a one-layer or multi-layer polymer matrix. In addition, systems are also available which comprise a layered structure having a reservoir, a membrane which controls the release of the drug, and an adhesive layer. Then there are other systems that have functional features which are even more specialised.

Almost all of the transdermal therapeutic systems that are commercially available today contain the drug in dissolved form. In the vast majority of these systems, the content of drug in each layer is below the saturation solubility at room temperature of the drug in the corresponding layer so that the drug does not crystallise. If the content of drug in a layer were to be above the saturation solubility of the drug in this layer, there would be a risk of crystallisation of the drug. Linked with this instability would be a reduced thermodynamic activity of the drug, which leads to a reduced release of the drug from the TTS.

One of the problems when developing transdermal therapeutic systems is thus that they are supposed to have the highest possible concentration of drug in order to be able to achieve a high delivery of the drug with a comparatively small release area, but on the other hand the stability of a TTS over a long storage period, possibly of several years, must also be ensured. In particular, the risk of a crystallisation of the drug in the TTS owing to its supersaturation is to be avoided. The dilemma when developing transdermal therapeutic systems is thus to use the highest possible concentration of drug without having to run the risk of a crystallisation of the drug in the TTS.

Numerous solutions have been proposed for stabilising TTS supersaturated with drug and for preventing crystallisation of the drug contained therein. Various excipients in particular seem suitable therefor, which are added to the TTS, in particular to the matrix containing the drug, during the production thereof.

Described in EP 0 391 172 A1 are transdermal therapeutic systems having a layered structure that is composed of a backing layer which is impermeable to active substances, a matrix with islands distributed therein which contain the drug, and a layer controlling the access of skin moisture. The matrix is based on a basic material, for example silicone polymers, which is permeable to water vapour, is substantially water insoluble and is largely free of active substance. The islands distributed in the basic material of the matrix are based on a water-soluble or water-swellable material such as polyvinyl alcohol or polyvinylpyrrolidone.

Disclosed in EP 0 481 443 A1 are transdermal therapeutic preparations having an improved storage stability in respect of the physical-chemical structure of the active substance. These preparations comprise a rigid backing layer, a polymeric matrix, and a removable protective layer, said polymeric matrix consisting of a polymer film in which particles are distributed that are loaded with active substance and a compound which improves the absorption of the active substance by the skin. The particles are microporous particles or polymeric microspheres, for example of cross-linked polyvinylpyrrolidone.

WO 01/01967 A1 discloses transdermal therapeutic systems based on polysiloxane, which contain microreservoirs comprising the active substance and an ambiphilic solvent. Cited as examples of ambiphilic solvents are 1,3 butanediol, dipropylene glycol, tetrahydrofurfuryl alcohol, diethylene glycol dimethyl ether, diethylene glycol monoethyl ether, diethylene glycol monobutyl ether, propylene glycol, dipropylene glycol, carboxylic acid esters of triethylene and diethylene glycol as well as polyoxyethylated fatty alcohols of 6-18 C atoms.

The laid-open document WO 01/68060 A2 describes transdermal therapeutic systems of the matrix type comprising a backing layer that is impermeable to active substances, a removable protective layer, and an active substance-containing matrix based on hydrophobic polymers, with the active substance having a melting point above room temperature and being present in a concentration exceeding the saturation solubility for at least part of the period of application of the TTS. These transdermal therapeutic systems are characterised in that a polyacrylate polymer is added to the hydrophobic base polymers of the active substance matrix, and/or the matrix layer containing the hydrophobic polymers is provided with a self-adhesive skin contact layer based on polyacrylates. In addition to the polyacrylates, mixtures of polyacrylates with other hydrophilic polymers such as polyvinylpyrrolidone or copolymers of vinylpyrrolidone with vinyl acetate can also be used.

Disclosed in WO 95/18603 A1 are transdermal devices containing polyvinylpyrrolidone as a solubility enhancer. A mixture of at least three polymers, for example one or more polysiloxanes, a polyacrylate and a water-soluble polyvinylpyrrolidone, leads, in combination with the active substance, to a pressure-sensitive adhesive preparation for a transdermal therapeutic system. The soluble polyvinylpyrrolidone increases the solubility of the active substance without having a negative effect on the adhesive properties or the release of the active substance from the pressure-sensitive adhesive preparation.

Described in WO 2011/076879 is a transdermal therapeutic system which comprises a solid dispersion consisting of the drug rotigotine and polyvinylpyrrolidone in a silicone pressure-sensitive adhesive mixture, wherein rotigotine and the polyvinylpyrrolidone can be present in a plurality of microreservoirs. Transdermal therapeutic systems comprising microreservoirs are furthermore already known from WO 2004/012719 and WO 2004/012730.

In spite of the numerous solution approaches, the problem of unstable, supersaturated systems has not been fundamentally solved since formulations in which the drug crystallises repeatedly appear in the transdermal therapeutic systems which are available on the market.

Furthermore, a transdermal administration has by no means been made available to all drugs since it has to date not been possible to develop for every drug systems having a sufficiently high concentration of drug or stable supersaturated systems.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

It is further noted that the invention does not intend to encompass within the scope of the invention any previously disclosed product, process of making the product or method of using the product which meets the written description and enablement requirements of the USPTO (35 U.S.C. 112, first paragraph) or the EPO (Article 83 of the EPC), such that applicant(s) reserve the right to disclaim, and hereby disclose a disclaimer of, any previously described product, method of making the product, or process of using the product.

### SUMMARY OF THE INVENTION

The object of the present invention was therefore to provide a transdermal therapeutic system which, with regard to the stable modification of a drug, is supersaturated but nevertheless stable when stored and does not exhibit any spontaneous recrystallisation of the drug. Based on pharmaceutical experiences with numerous drugs, the scope of implementation of the present invention is restricted to those drugs having a kinetically delayed spontaneous crystallisation rate.

The object is solved by providing a transdermal therapeutic system (TTS) comprising a backing layer that is impermeable to active substances, a matrix consisting of one, two or more layers, and a removable protective layer, characterised in that the matrix layer(s) or at least one of the matrix layers comprises/comprise at least one pressure-sensitive adhesive, at least one drug as well as particles of cross-linked polyvinylpyrrolidone, said pressure-sensitive adhesive being a hot melt pressure-sensitive adhesive.

### DETAILED DESCRIPTION OF EMBODIMENTS

It is to be understood that the figures and descriptions of the present invention have been simplified to illustrate elements that are relevant for a clear understanding of the present invention, while eliminating, for purposes of clarity, many other elements which are conventional in this art. Those of ordinary skill in the art will recognize that other elements are desirable for implementing the present invention. However, because such elements are well known in the art and because they do not facilitate a better understanding of the present invention, a discussion of such elements is not provided herein.

The present invention will now be described in detail on the basis of exemplary embodiments.

The transdermal therapeutic systems of the present invention are fundamentally hot melt TTS based on hot melt pressure-sensitive adhesives that are produced without solvent.

The transdermal therapeutic systems according to the invention are furthermore plasters for application to the skin of a patient.

The terms “transdermal therapeutic system”, “TTS” and “plaster” are used synonymously within the context of the present invention.

The drug(s) and the particles of cross-linked polyvinylpyrrolidone form an inner phase and are present in dispersed form in the pressure-sensitive adhesive as the outer phase of the transdermal therapeutic systems according to the invention. The pressure-sensitive adhesive therefore has the function of a dispersion medium.

Understood as hot melt pressure-sensitive adhesives within the meaning of the present invention are pressure-sensitive, hot meltable adhesives which form a bond with a non-adhesive surface upon the application of pressure and which soften under the influence of temperature so that they can be processed into the transdermal therapeutic systems according to the invention. Hot meltable pressure-sensitive adhesives for use in the present invention can consist of both one pressure-sensitive adhesive and a mixture of different pressure-sensitive adhesives.

According to the invention, those hot melt pressure-sensitive adhesives which contain silicone polymers formed from polydimethylsiloxane are preferred. In general, the silicone pressure-sensitive adhesives that can be used according to the invention are hot melt pressure-sensitive adhesives produced on the basis of silicone polymers, which preferably contain at least 50% by weight, in particular 60-95% by weight, particularly preferred 75-90% by weight of silicone polymer(s), such as, for example, silicone polymers having a polydimethylsiloxane structure or a polydimethyldiphenyl siloxane structure.

According to a preferred embodiment, the transdermal therapeutic systems according to the invention comprise as the pressure-sensitive adhesive at least one of the above-described silicone hot melt pressure-sensitive adhesives, which furthermore preferably contains a silicone polymer formed from polydimethylsiloxane.

Silicone hot melt pressure-sensitive adhesives for use in the present invention may additionally contain silicone oils and/or other softeners (plasticisers).

Additionally, mixtures or condensates of silicone resins and polyorganosiloxanes also come into consideration. Amine-resistant silicone pressure-sensitive adhesives are furthermore preferred, which are characterised in that they do not contain any or only contain a few free silanol functions since the Si—OH groups were alkylated.

According to a preferred embodiment, a hot melt pressure-sensitive adhesive is used which softens when heated and thereby achieves a viscosity that is suitable for incorporating one, two or more drugs in solid form and the particles of cross-linked PVP, as well as for application by means of slot extrusion or coating, and which, following cooling, is once again present in a non-flowable state.

The softening temperature of suitable hot melt pressure-sensitive adhesives lies in the range of between 50 and 200° C., preferred between 75 and 170° C., and particularly preferred between 100 and 150° C.

Corresponding hot melt pressure-sensitive adhesives preferably have a dynamic viscosity in the softened state of at most 150 Pa·s, preferably of at most 120 Pa·s, particularly preferred of less than 100 Pa·s, further preferred of less than 80 Pa·s, and in particular preferred of less than 60 Pa·s.

A suitable silicone hot melt pressure-sensitive adhesive for use in the present invention is, for example, the hot melt pressure-sensitive adhesive BIO-PSA 7-4560 of Dow Corning.

In addition to silicone hot melt pressure-sensitive adhesives, other hot melt pressure-sensitive adhesives also come into consideration as pressure-sensitive adhesives provided that they—like the silicone hot melt pressure-sensitive adhesives—have a high active substance diffusibility and a low active substance solubility, such as, for example, styrene-block-copolymer-based hot meltable adhesives (“SXS pressure-sensitive adhesives”) or ethylene-vinyl-acetate-copolymer-based hot meltable adhesives (“EVA pressure-sensitive adhesives”).

The active substance solubility in the hot melt pressure-sensitive adhesives suitable for use in the present invention (i.e. the solubility of a drug in a suitable hot melt pressure-sensitive adhesive) is preferably 0-2% by weight and preferred 0-0.5% by weight.

Hot melt pressure-sensitive adhesives and in particular silicone hot melt pressure-sensitive adhesives that are suitable for use in the present invention and such as are described above are fundamentally known to the person skilled in the art and are commercially available.

Particles of cross-linked polyvinylpyrrolidone, i.e. cross-linked polyvinylpyrrolidone in particulate form, are known to the person skilled in the art and are commercially available. These are generally particles having an average particle size (grain size) of 5 μm to 500 μm, with particles having an average particle size of 5-100 μm being preferred. If it is not specified by the manufacturer, the average particle size can be ascertained in a manner known to the person skilled in the art (for example by way of particle size determination using laser diffraction). However, the particles of cross-linked polyvinyl-pyrrolidone should fundamentally not be larger in their longest extension than the thickness of the layer or the individual layers of the matrix of the transdermal therapeutic systems according to the invention, which contains/contain at least one drug and the particles of cross-linked polyvinylpyrrolidone.

Cross-linked polyvinylpyrrolidone is characterised in that it is insoluble in water as well as in organic solvents.

In the TTS according to the invention, the cited polyvinylpyrrolidone particles are dispersed in the hot melt pressure-sensitive adhesive, which forms the drug-containing matrix layer(s).

Owing to the water insolubility of cross-linked polyvinylpyrrolidone, the integrity of the polyvinylpyrrolidone particles dispersed in the TTS according to the invention is still ensured even if the patient sweats or begins to sweat whilst wearing one, two or more of the TTS according to the invention on their skin.

The total amount of cross-linked polyvinylpyrrolidone in the respective layers of the matrix of the transdermal therapeutic systems according to the invention, which comprise at least one hot melt pressure-sensitive adhesive, at least one drug as well as particles of cross-linked polyvinylpyrrolidone, is 1-25% by weight, preferably 2-15% by weight and particularly preferred 5-10% by weight.

According to a preferred embodiment, the drug-containing matrix layers of the transdermal therapeutic systems according to the invention comprise at least one hot melt pressure-sensitive adhesive, in particular a silicone hot melt pressure-sensitive adhesive, at least one drug, and particles of cross-linked polyvinylpyrrolidone having an average particle size of 5-500 μm, preferably 5-100 μm.

The drug content of the transdermal therapeutic systems according to the invention is preferably 5-25% by weight, particularly preferred 10-20% by weight, and in particular 15-20% by weight, in each case based on the drug-containing matrix layer(s) of the transdermal therapeutic systems according to the invention. It is furthermore preferred according to the invention that the drug concentration is such that the matrix layer(s) is/are supersaturated in respect of the stable modification of the drug(s).

The mass ratio of drug to polyvinylpyrrolidone in the transdermal therapeutic systems according to the invention is preferably in the range of 10:1 to 1:10.

According to a further preferred embodiment, the drug-containing matrix layer(s) of the transdermal therapeutic systems according to the invention comprises/comprise at least one hot melt pressure-sensitive adhesive, in particular a silicone hot melt pressure-sensitive adhesive, at least one drug, and particles of cross-linked polyvinylpyrrolidone having an average particle size of 5-500 μm, preferably 5-100 μm, with the mass ratio of drug(s) to polyvinylpyrrolidone being in the range of 10:1 to 1:10.

Coming into consideration as drugs for the transdermal therapeutic systems according to the invention are pharmaceutical active substances (as well as the salts thereof), preferably those which have a low tendency for spontaneous crystallisation. Particularly suitable are drugs selected from the group of pharmaceutical active substances comprising estradiol, preferably anhydrous estradiol, as well as buprenorphine, rotigotine, rivastigmine, scopolamine, granisetron, lerisetron, ramosetron, ondansetron and pramipexole, as well as pharmaceutically acceptable salts of the aforementioned substances.

According to a preferred embodiment, anhydrous estradiol, scopolamine or rotigotine are used as the drug in the transdermal therapeutic systems according to the invention.

The respective drugs are present in the finished transdermal therapeutic systems according to the invention in a non-crystalline form.

Within the context of the present invention, the term “non-crystalline form” means that the respective drug can be present both in the form of a solid solution and in amorphous form as well as in both forms at the same time.

It is assumed, without being bound by this theory, that the drug(s) is/are present in the dispersion medium/outer phase, i.e. in the pressure-sensitive adhesive, in a molecularly dispersed form, and that a non-crystalline form of the drug(s) is reversibly bound to the particles of cross-linked PVP, with the drug(s) bound to the particles of cross-linked PVP forming an inner phase in the form of a plurality of microreservoirs.

This does not rule out and would normally even imply that a certain portion of the drug(s) is present in dissolved form in the dispersion medium up to its saturation concentration.

Within the framework of this description, “microreservoirs” are to be understood as particulate compartments which are spatially and functionally separate, which consist of a mixture of drug and cross-linked PVP, and which are dispersed in the pressure-sensitive adhesive of the transdermal therapeutic systems according to the invention. The transdermal therapeutic systems according to the invention preferably contain 10³ to 10⁹ and particularly preferred 10⁶ to 10⁹ microreservoirs per cm² of their surface.

The maximum diameter of the microreservoirs is less than the thickness of the drug-containing matrix layer(s) of the transdermal therapeutic systems according to the invention and is preferably up to 70% of the thickness of the matrix layer(s) and particularly preferred 5 to 60% of the thickness of the matrix layer(s). For an example thickness of the matrix layer(s) of 50 μm, this corresponds to a maximum diameter of the microreservoirs in the range of preferably up to 35 μm.

The term “maximum diameter” refers to the diameter of the microreservoirs which is the largest within the three spatial dimensions (x, y or Z dimension). It is clear to the person skilled in the art that in the case of spherical micro-reservoirs, the maximum diameter corresponds to the diameter of the microreservoirs. In the case of microreservoirs that are not spherical, i.e. that are present in different geometric forms, the extension thereof in the respective x, y and z dimensions can differ greatly.

According to a particularly preferred embodiment, the average diameter of the microreservoirs that are distributed in the drug-containing matrix layer(s) of the transdermal therapeutic systems according to the invention is in the range of 1 to 40%, more preferred in the range of 1 to 20% of the thickness of the matrix layer(s). For an example thickness of the matrix layer(s) of 50 μm, this corresponds to an average diameter of the microreservoirs in the range of preferably 0.5 to 20 μm.

The term “average diameter” refers to the mean value of the x, y and z average diameter of all microreservoirs.

The maximum and average diameters of the microreservoirs as well as the number of microreservoirs per surface can be determined as follows. After removing the removable protective layer, the surface of the respective transdermal therapeutic systems is examined using a light microscope (for example using a Leica microscope of the type DM/RBE, equipped with a camera of the type Basler A 113C). The measurement is carried out by way of random analysis with polarised light using a microscope at 200× magnification. An image analysis can be carried out, for example using the software Nikon LuciaDi, version 4.21, which leads to average and maximum diameters for each sample.

In a preferred embodiment, the transdermal therapeutic systems according to the invention contain one drug or two drugs, and particularly preferred at least one drug. In a further embodiment, the transdermal therapeutic systems according to the invention can also contain two or more drugs.

With regard to the solution of the object forming the basis for the present invention, the occurrence of nuclei of crystallisation during the production process should be prevented as much as possible. The production of the transdermal therapeutic systems according to the invention therefore preferably occurs under the application of heat.

This is accomplished according to the invention in that during the production process, the temperature of the drug-containing hot melt pressure-sensitive adhesive mass or the drug-containing pressure-sensitive matrix layer(s) of the transdermal therapeutic systems, more specifically the plasters, according to the invention, which are produced therefrom is at least temporarily above the melting point or the melting range of the stable modification of the drug(s) to be incorporated. This heat treatment can be carried out during production of the drug-containing hot melt pressure-sensitive adhesive mass and/or during coating of the backing layer which is impermeable to active substances.

The mentioned temperature is preferably at least 5-10° C., particularly preferred at least 10° C. and in particular 5 to 50° C., and preferred 10 to 25° C. above the melting point or the melting range of the stable modification of the respective drug, whereby within the framework of the present invention, the reference point in the case of the melting range is the maximum value thereof.

The term “stable modification of the respective drug” relates in particular to the thermodynamically stable modification of the respective drug. Within the framework of the present invention, the term “stable modification of the respective drug” furthermore includes both the crystalline form, including various crystal modifications (insofar as they are present), and the amorphous form of a drug.

It is sufficient for the heat treatment described above if the cited temperature is reached or more specifically maintained for a short time, preferably for a duration of at least 1 min, in particular 1 min to 10 min.

The stable modifications of the respective drugs as well as the melting points or melting ranges of these stable modifications are known to the person skilled in the art or can be found in expert literature.

In the following, the melting points or melting ranges of the stable modifications of some of the drugs which can be used according to the invention are provided as examples:
Estradiol: 173-179° C.;
Buprenorphine: 209° C.;
Rotigotine: 97° C.;
Rivastigmine tartrate: 123-125° C.;
Scopolamine hydrobromide: 195° C.;
Granisetron hydrochloride: 290-292° C.;
Ondansetron hydrochloride: 178.5-179.5° C.;
Pramipexole dihydrochloride: 296-298° C.

The success according to the invention, i.e. the prevention of the recrystallisation of the drug in a matrix layer of a transdermal therapeutic system that is supersaturated with drug, occurs in particular in the case of the combination of the formulations according to the invention with the heat treatment described above.

In particular suitable for the application of the production method according to the invention are such drugs that have a melting point or melting range that lies between 20° C. and 350° C. Particularly suitable are drugs having a melting point or melting range of between 30° C. and 200° C.

Whether or not a specific drug has a low tendency for spontaneous crystallisation and is thus particularly suitable for the present invention can be determined by means of a preliminary experiment, in which the saturation solubility of the drug in a solvent, preferably ethanol, is first of all determined.

For this purpose, a suspension of the stable modification of the drug in the solvent is stirred for 24 hours at 25° C. such that an equilibrium is established. After filtration, the content of drug in the supernatant is determined using a suitable analytical method that is known to the person skilled in the art. A double concentrated solution is then produced using the same solvent by weighing a corresponding amount of drug into the solvent. The initially generated suspension is heated until all of the residues of the crystalline drug have dissolved in the solvent. Solvent evaporated during this time is to be replaced and the solution is then to be cooled.

A glass ampoule having a volume of a maximum of 1 ml is filled with the solution and the solution is heated again in the sealed ampoule for at least 10 minutes to a temperature that is at least 10° C. above the melting point (or above the melting range) of the stable modification of the drug, and is then stored at 25° C. for 24 hours. Drugs that stay dissolved in the liquid solvent under these conditions exhibit a low tendency for spontaneous crystallisation and are thus particularly suitable for being processed into a TTS according to the invention using the method according to the invention.

In order to produce the transdermal therapeutic systems according to the invention, a method is preferred in which the drug-containing hot melt pressure-sensitive adhesive mass for the matrix layer(s) is produced without solvent. For this purpose, the hot melt pressure-sensitive adhesive is heated until solid cross-linked polyvinylpyrrolidone, which is present in the form of particles, and the required amount of one, two or more and preferably at least one crystalline or amorphous drug can be added and dispersed in the hot melt pressure-sensitive adhesive mass by means of kneading or stirring. A silicone hot melt pressure-sensitive adhesive is preferably used as the hot melt pressure-sensitive adhesive.

The hot melt pressure-sensitive adhesive is thereby heated to a temperature that is 5-20° C. and preferably approximately 10° C. above the softening temperature of the hot melt pressure-sensitive adhesive mass and is at the same time below the melting point or the melting range of the stable modification of the drug(s) to be incorporated.

The selection of one of the above-described hot melt pressure-sensitive adhesives having a suitable melting range for use in the transdermal therapeutic systems according to the invention thus also depends on the drug(s) to be incorporated and the respective melting points or melting ranges thereof.

In addition, pharmaceutical excipients which are known to the person skilled in the art, for example permeation enhancers, softeners, antioxidants and the like, may be added to the hot melt pressure-sensitive adhesive mass.

The drug-containing hot melt pressure-sensitive adhesive mass that is obtained in this manner is applied to a suitable film- or sheet-like polymer carrier using a suitable method, for example by way of slot extrusion or by coating using a doctor's knife in the form of a roller or a coating box. In a preferred embodiment, the film- or sheet-like polymer carrier forms the backing layer of the finished TTS, which is impermeable to active substances.

Immediately after coating of the polymer carrier, the applied hot melt pressure-sensitive adhesive mass cools and, since it is spontaneously adhesive, can be covered with a further polymer film or a further polymer sheet. It is, however, also possible to apply further drug-containing coatings to the already coated polymer carrier before covering with the further polymer film or the further polymer sheet.

According to a preferred embodiment, the further polymer film or the further polymer sheet forms the removable protective layer of the finished TTS.

The individual transdermal therapeutic systems according to the present invention are formed by punching them out of the obtained laminate that consists of one, two or more drug-containing matrix layers between two layers of polymer sheet or polymer film, and these can be packaged in individual packagings.

During the production of the transdermal therapeutic systems of the invention according to the method described above, a heat treatment is additionally carried out, in which the temperature during production of the drug-containing hot melt pressure-sensitive adhesive mass and/or the coating of the polymer carrier is raised at least temporarily above the melting point or the melting range of the stable modification of the respective drug(s).

The heat treatment in the above-described method preferably occurs at a temperature that is at least 5-10° C., particularly preferred at least 10° C., and in particular 5-50° C., and preferred 10-25° C. above the melting point or the melting range of the stable modification of the respective drug(s).

It is sufficient for the heat treatment if the cited temperature is reached or more specifically maintained for a short time, preferably for a duration of at least 1 min, in particular 1 min to 10 min.

Also preferred is a transdermal therapeutic system according to the above, which is characterised in that during production of the drug-containing matrix layer(s) and/or during coating of the backing layer of the transdermal therapeutic system, which is impermeable to active substances, a heat treatment was carried out in which the temperature was above the melting point or the melting range, preferably at least 5-10° C., particularly preferred at least 10° C., and in particular 5-50° C., and preferred 10-25° C. above the melting point or the melting range of the stable modification of the drug(s).

In a preferred embodiment, the method for producing the transdermal therapeutic systems according to the invention comprises the steps of
a) producing a drug-containing pressure-sensitive adhesive mass comprising at least one pressure-sensitive adhesive, at least one drug, and particles of cross-linked polyvinylpyrrolidone,
said pressure-sensitive adhesive being a hot melt pressure-sensitive adhesive, and the drug and the polyvinylpyrrolidone particles being dispersed in the hot melt pressure-sensitive adhesive;
b) coating a film- or sheet-like polymer carrier with the drug-containing hot melt pressure-sensitive adhesive mass;
   and is characterised in that during production, a heat treatment is carried out, in which the temperature is above the melting point or the melting range of the stable modification of the drug(s).

The temperature during the heat treatment is preferably at least 10° C. above the melting point or the melting range of the stable modification of the drug(s).

In a further preferred embodiment, the method comprises the following additional steps:
c) covering the coating with a polymer sheet or a polymer film,
d) punching out individual transdermal therapeutic systems,
e) packaging the individual transdermal therapeutic systems.

It is particularly preferred for the heat treatment in the above-described method to occur during production of the drug-containing pressure-sensitive adhesive mass and/or during coating of the film- or sheet-like polymer carrier.

In a particularly preferred embodiment, the hot melt pressure-sensitive adhesive used in the above-described method is a silicone hot melt pressure-sensitive adhesive, which furthermore preferably contains a silicone polymer formed from polydimethylsiloxane.

Furthermore preferred is a transdermal therapeutic system that was produced by means of the method described above.

### OPERATIVE EXAMPLES

The subject matter of the present invention is elucidated in more detail below, using examples, without any intention that the subject matter of the invention should be confined to these exemplary embodiments.

### Example

A hot meltable adhesive mass (hot melt pressure-sensitive adhesive mass) based on a silicone polymer is heated to a temperature that is 10° C. above the softening temperature of the hot melt pressure-sensitive adhesive. 15% by weight of cross-linked polyvinylpyrrolidone having an average particle size of 20 μm and any necessary stabilisers are then added. The polyvinylpyrrolidone particles are dispersed in the hot melt pressure-sensitive adhesive mass by way of kneading. 10% by weight of a drug in solid form is subsequently added and is also distributed in the hot melt pressure-sensitive adhesive mass by way of kneading. Finally, the hot melt pressure-sensitive adhesive mass is heated to a temperature that is 10° C. above the melting temperature or the melting range of the stable modification of the drug.

The drug-containing hot melt pressure-sensitive adhesive mass obtained in this manner is extruded via a slot nozzle at a thickness of 100 μm onto a sheet-like polymer carrier in the form of a polyester sheet having a thickness of 20 μm, and the extruded layer is covered with a suitable protective sheet. Individual TTS are then punched out of the overall laminate obtained in this manner and are packaged in sealed pouches.

While this invention has been described in conjunction with the specific embodiments outlined above, it is evident that many alternatives, modifications, and variations will be apparent to those skilled in the art. Accordingly, the preferred embodiments of the invention as set forth above are intended to be illustrative, not limiting. Various changes may be made without departing from the spirit and scope of the intentions as defined in the following claims.

1. A transdermal therapeutic system (TTS) comprising a backing layer that is impermeable to active substances, a matrix consisting of one, two or more layers, and a removable protective layer, wherein the matrix layer(s) or at least one of the matrix layers comprises/comprise at least one pressure-sensitive adhesive, at least one drug, as well as particles of cross-linked polyvinylpyrrolidone, said pressure-sensitive adhesive being a hot melt pressure-sensitive adhesive.

2. The transdermal therapeutic system according to claim 1, wherein the hot melt pressure-sensitive adhesive is a silicone hot melt pressure-sensitive adhesive containing a silicone polymer formed from polydimethylsiloxane.

3. The transdermal therapeutic system according to claim 1, wherein the average particle size of the particles of cross-linked polyvinylpyrrolidone is 5-500 μm.

4. The transdermal therapeutic system according to claim 1, wherein the drug(s) has/have a low tendency for spontaneous crystallisation.

5. The transdermal therapeutic system according to claim 1, wherein the drug(s) is/are selected from the group comprising anhydrous estradiol as well as buprenorphine, rotigotine, rivastigmine, scopolamine, granisetron, lerisetron, ramosetron, ondansetron and pramipexole, as well as pharmaceutically acceptable salts thereof.

6. The transdermal therapeutic system according to claim 1, wherein the mass ratio of drug(s) to polyvinylpyrrolidone is in the range of 10:1 to 1:10.

7. The transdermal therapeutic system according to claim 1, wherein the drug(s) and the particles of cross-linked polyvinylpyrrolidone are present in a plurality of microreservoirs.

8. The transdermal therapeutic system according to claim 1, wherein during production of the drug-containing matrix layer(s) and/or during coating of the backing layer of the transdermal therapeutic system, which is impermeable to active substances, a heat treatment was carried out in which the temperature was above the melting point or the melting range, of the stable modification of the drug(s).

9. A method for the production of a transdermal therapeutic system comprising the steps of
a) producing a drug-containing pressure-sensitive adhesive mass comprising at least one pressure-sensitive adhesive, at least one drug, and particles of cross-linked polyvinylpyrrolidone,
said pressure-sensitive adhesive being a hot melt pressure-sensitive adhesive, and the drug and the polyvinylpyrrolidone particles being dispersed in the hot melt pressure-sensitive adhesive; and
b) coating a film- or sheet-like polymer carrier with the drug-containing pressure-sensitive adhesive mass;
wherein during production, a heat treatment is carried out, in which the temperature is above the melting point or the melting range of the stable modification of the drug(s).

10. The method according to claim 9, wherein the temperature during the heat treatment is at least 10° C. above the melting point or the melting range of the stable modification of the drug(s).

11. The method according to claim 9, further comprising:
c) covering the coating with a polymer sheet or a polymer film,
d) punching out individual transdermal therapeutic systems, and
e) packaging the individual transdermal therapeutic systems.

12. The method according to claim 9, wherein the heat treatment occurs during production of the drug-containing pressure-sensitive adhesive mass and/or during coating of the film- or sheet-like polymer carrier.

13. The method according to claim 9, wherein the hot melt pressure-sensitive adhesive is a silicone hot melt pressure-sensitive adhesive, which contains a silicone polymer formed from polydimethylsiloxane.

14. A transdermal therapeutic system produced by means of the method as claimed in claim 9.

15. The transdermal therapeutic system according to claim 1, wherein the average particle size of the particles of cross-linked polyvinylpyrrolidone is 5-100 μm.

16. The transdermal therapeutic system according to claim 8 wherein the heat treatment was carried out at a temperature of at least 10° C. above the melting point or melting range of the stable modification of the drug(s).
